(19) United States
(12) Patent Application Publication
(10) Pub. No.: US 2007/0014447 A1
(43) Pub. Date: Jan. 18, 2007

(54) Method for locating brain lesion

(76) Inventor: Otakar R. Hubschmann, Short Hills, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/171,168

(22) Filed: Jun. 30, 2005

(51) Int. Cl. G06K 9/00; A61B 5/05

(52) U.S. Cl. 382/128; 600/407

(57) ABSTRACT

A surgeon utilizes a brain scan image to locate a brain lesion and to plan the operation to treat it. Distance measurements representing the location of the lesion are derived from various views of a brain scan image. In the operating room, he transfers these distance measurements onto the patient's cranium in a “warped” or curvilinear Cartesian coordinate system defined by intersecting orthogonal lines extending along the surface of the patient's cranium. As a result, the lesion is localized and the surgeon may establish the appropriate location and orientation of a bone flap to be used to access the lesion.

### BACKGROUND OF THE INVENTION

In performing cranial surgery, it is the common practice to form a bone flap in the patient's cranium so as to gain access to the brain. While standard bone flaps may be used during neurosurgery for specific lesions, such as for an aneurysm clipping and certain types of skull base surgery procedures, removal of most cranial lesions requires a specific “customized” flap. Requirements of present day neurosurgery make it imperative that the flap be accurate and as small as possible. This is typically achieved by frame-based or frameless stereotaxi.

Frame-based stereotaxi involves the placement of a frame on the patient's head, which provides a spatial frame of reference and the acquisition of a brain scan image, such as a Computed Tomography (CT) scan or Magnetic Resonance imaging (MRI). Frameless stereotaxi requires the placement of fiducial markers on the patient's head. Another scan is necessary prior to any surgical procedure, which requires transferring the patient between the operating room and the radiology center. Stereotaxi is therefore time-consuming, often doubling the operating room time. It also requires coordination with the radiology center, and it often requires the presence of a technical support person from the company that manufactures the equipment. In addition, the equipment and systems used to perform stereotaxi are cumbersome, not user friendly, and quite expensive.

A need therefore exists for a simpler and less expensive method that could be deployed rapidly to locate brain lesions, and to form a flap quickly and accurately, without the need for complex equipment or technical support and without the need to leave the operating room.

### SUMMARY OF THE INVENTION

In accordance with one aspect of the present invention, a surgeon utilizes a brain scan image to locate a brain lesion and to plan the operation to treat it. In the operating room, he transfers distance measurements derived directly from the brain scan onto the patient's cranium, thereby establishing the appropriate location and orientation of the bone flap.

### BRIEF DESCRIPTION OF THE DRAWING

The foregoing brief description, as well as further objects, features, and advantages of the present convention will be understood more completely from the following description of a presently preferred, but nonetheless illustrative, embodiment in accordance with the present convention, with reference being had to the accompanying drawings, in which:

FIG. 1 depicts a saggital MRI of the patient's head showing a brain lesion;

FIG. 2 depicts a coronal MRI of the same patient's head to;

FIG. 3 illustrates a method for locating a brain lesion using a “warped” Cartesian coordinate system;

FIG. 4 illustrates and alternate method for locating a brain lesion using a “warped” Cartesian coordinate system; and

FIG. 5 illustrates a bone flap having been outlined on the patient's scalp after the brain lesion has been located; and

FIG. 6 is a block diagram of a computer system useful in measuring distances in the MRIs.

### DETAILED DESCRIPTION OF THE PREFERRED EMBODIMENTS

As explained above, a surgeon utilizes a brain scan image to locate a brain lesion and to plan the operation to treat it. In the operating room, he transfers distance measurements derived directly from the brain scan onto the patient's cranium, thereby establishing the appropriate location and orientation of the bone flap.

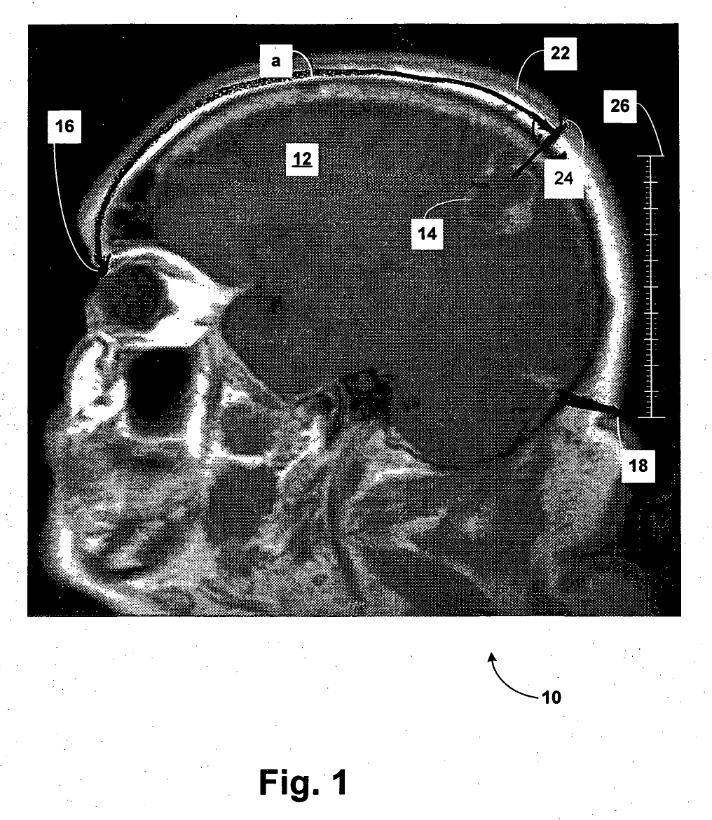

FIG. 1 is a saggital MRI 10 of a patient's head showing the brain which has a lesion 14. The figure also shows the patient's nasion 16 (the recess above the nose) and inion 18 (the recess at the base of the skull). As may be seen, MRI 10 has a scale 26, which is typical. The scale 26 may be in the centimeters (large gradations), indicating the length of a centimeter on the patient's head.

As an initial step, the surgeon measures along the surface of the cranium 22 the distance a, the distance measured on the MRI from the nasion 16 to a point overlying the center of the lesion 14. This measurement ends at the point 24. The depth of the lesion in this view is determined by dropping a perpendicular to the cranial surface from point 24.

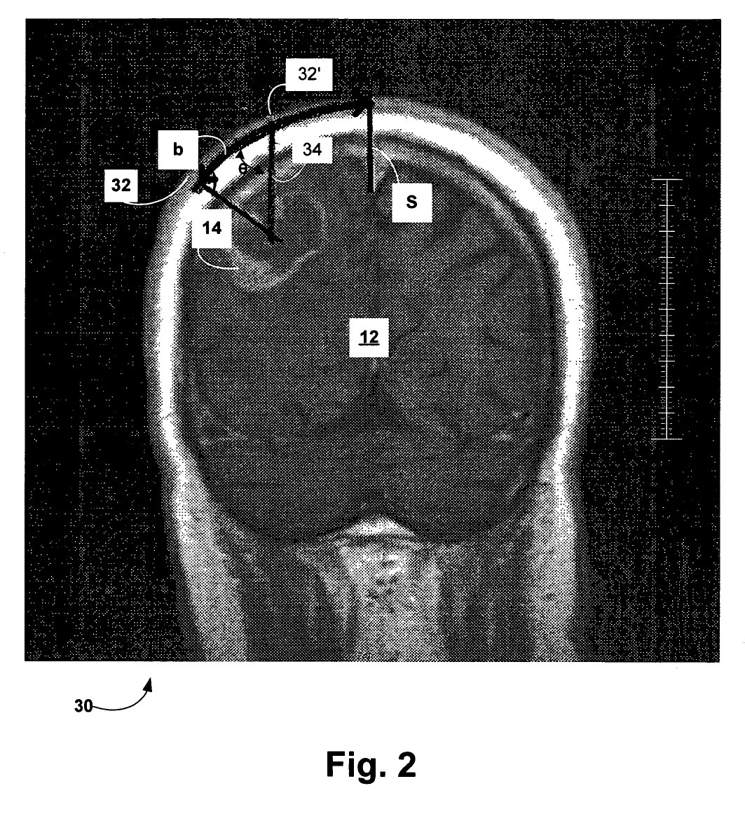

FIG. 2 is a coronal MRI of the same patient's head. As a second step, the surgeon measures the distance b from the saggital plane S to the center of the lesion 14 along the surface of the cranium. This measurement ends at point 32. The depth of the lesion in this view is determined by dropping the perpendicular to the cranial surface from a point 32. This figure also illustrates another possible approach to the lesion 14. For example, if the tumor could not be approached from point 32 owing to the risk of damage to an important area of the brain, the lesion could be approached from point 32 prime along a line 34. In this case, line 34 is not normal to the surface of the cranium, so the surgeon would have to account for the angle θ when approaching the lesion.

Although MRIs conventionally have a linear scale 26, to previous measurements made by the surgeon were along the curved surface of the cranium. For example, manually operated devices have long been available which may be traced along any path on the surface of a map and will measure the length of the path. Typically, such devices include a small roller which is traced over the path and which can be calibrated to any scale on the map. Such devices are readily adapted to measuring distances along curvilinear paths on an MRI.

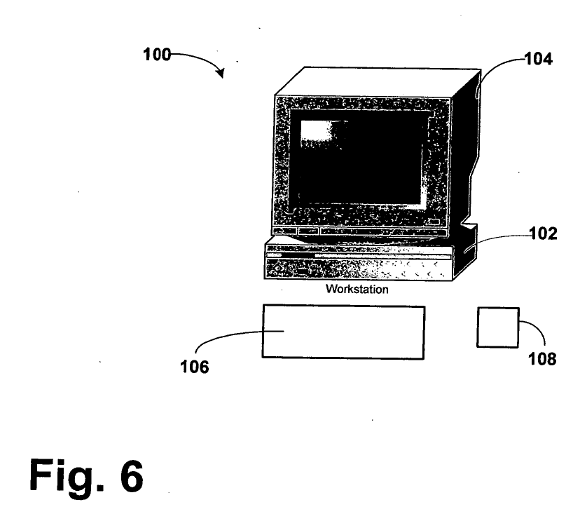

Preferably, the MRIs are transferred into a computer system 100 running a computer aided design program, as by means of the scanner 105, as shown schematically in FIG. 6. The system 100 also includes a computer 102, a display 104, and various additional input devices, such as the keyboard 106 and the pointing device 108. The computer runs a computer aided design program. Such programs are well-known, for example Photomodeler available from EOS Systems, Inc. Such programs are able to measure the length of the various curved lines, using the scales appearing on the MRIs for calibration purposes. Such a system could also measure the depth of the lesion in various views to compute its actual depth in three dimensions.

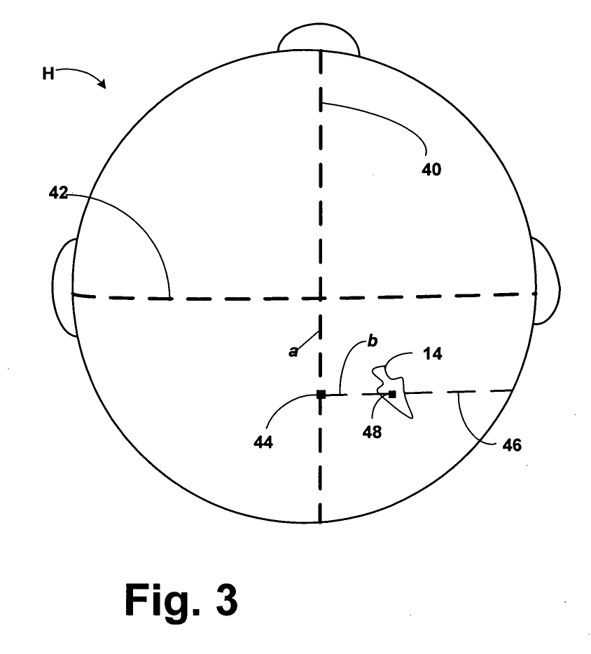

In the operating room, the surgeon makes use of the previously discussed measurements by first creating a “warped” Cartesian coordinate system on the patient's head. This is illustrated in the conceptual representation of the patient's head shown in FIG. 3. Specifically, the “y-axis” 40 of the coordinate system is defined by a saggital midline extending along the surface of the cranium. This y-axis is created by marking a line from the nasion to the inion on the patient's scalp. The x-axis of the coordinate system 42 is then created by marking a line on the patient's scalp between the ear canals.

Starting at the patient's nasion, the surgeon then lays out on the y-axis the distance a that was measured in the saggital MRI of FIG. 1. This results in the point 44. Starting at point 44, the surgeon then marks on the patient's scalp a line 46 which is perpendicular to the y-axis. Line 46 will also be parallel to the x-axis. Starting at the y-axis, the surgeon lays out on line 46 the distance b which was measured in the coronal MRI of FIG. 2. This results in the point 48, which is considered to overlie the lesion 14.

This procedure has been found to locate the lesion with an accuracy of about 1 cm.

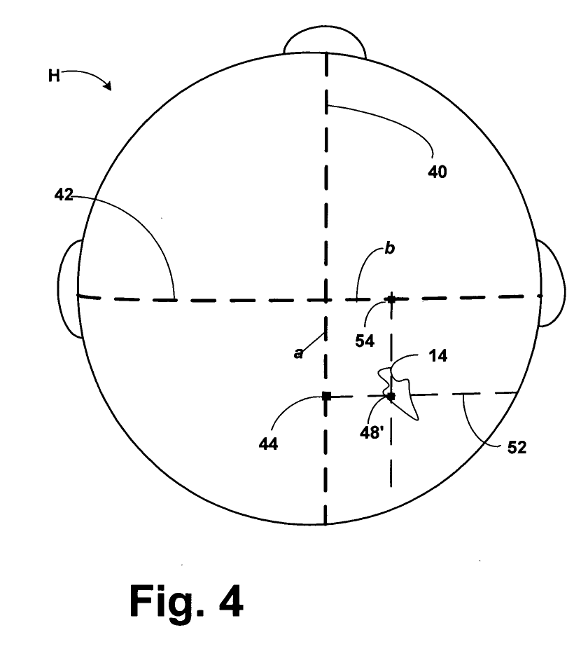

An alternative, similar procedure for locating point 48 may be preferred when the lesion is located close to the posterior or anterior regions of the head. This is illustrated in FIG. 4. The distance a laid out on the y-axis 40, as before. The distance b is then laid out on the x-axis 42 to produce a point 54. A line 52 is then drawn parallel to the x-axis and so as to pass through point 44 at the end of distance a, and a line 56 is drawn parallel to the y-axis and so as to pass through point 54. The intersection of lines 52 and 54 occurs at a point 48′, which is considered to overlie the lesion 14.

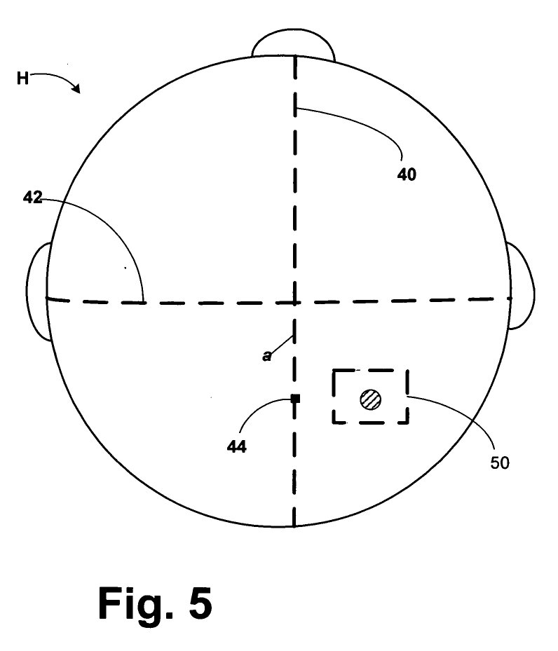

As shown in the conceptual view of FIG. 5, a bone flap 50 is then outlined at point 48 (or 48′).

As a final confirmation, after the bone flap 50 is formed and the dura of the cranium is exposed, the surgeon confirms the presence of the underlying lesion 14, as by ultrasound.

Although preferred embodiments of the invention has been disclosed for illustrative person purposes, those skilled in the art will appreciate that many additions, modifications, and substitutions are possible without departing from the scope and spirit of the invention as defined by the accompanying claims.

1. A method for locating a lesion in the brain of a patient, the method being performed with the aid of a computer system having a display showing a representation of a brain scan image illustrating features of the interior of the patient's cranium, the representation of the brain scan image including at least a saggital view and a coronal view, the method comprising the steps of:
in the saggital view, indicating two points defining, respectively, the location of a first anatomic landmark on the patient's head and the location of the lesion, causing the computer to measure the distance therebetween along the surface of the patient's cranium, to produce a first dimension;
in the coronal view, causing the computer to measure the distance along the surface of the patient's cranium from a substantially saggital midline to the lesion, to produce a second dimension; and
along a substantially saggital midline on the surface the patient's cranium, laying out the first dimension as a distance from the anatomic landmark, to arrive at a first point on the cranium; and a
laying out the second dimension along a line substantially perpendicular to the saggital midline, as a distance from the saggital midline to arrive at a target point corresponding substantially to the location of the lesion.

2. The method of claim 1 wherein the second dimension is measured from the first point.

3. The method of claim 2 a wherein the anatomic landmark is the patient's nasion.

4. The method of claim 2 wherein the landmark is the patient's inion.

5. The method of claim 2 is wherein the substantially saggital midline along which the laying out step is performed is a line which extends between the patient's nasion and inion.

6. The method of claim 2 further comprising establishing a substantially coronal midline on the surface of the patient's cranium as a line extending between the patient's ear canals, the second dimension being measure on the coronal midline to produce an intermediate point, the intermediate point being aligned with the first point.

7. The method of claim 6 a wherein the anatomic landmark is the patient's nasion.

8. The method of claim 6 wherein the landmark is the patient's inion.

9. The method of claim 6 is wherein the substantially saggital midline extends between the patient's nasion and inion.

10. The method of claim 1 wherein the anatomic landmark is the patient's nasion.

11. The method of claim 1 wherein the landmark is the patient's inion.

12. The method of claim 1 wherein the substantially saggital midline extends between the patient's nasion and inion.

13. The method of claim 1 further comprising establishing a substantially coronal midline on the surface of the patient's cranium as a line extending between the patient's ear canals, the second dimension being measure on the coronal midline to produce an intermediate point, the intermediate point being aligned with the first point.

14. The method of claim 13 wherein the anatomic landmark is the patient's nasion.

15. The method of claim 13 wherein the landmark is the patient's inion.

16. The method of claim 13 is wherein the substantially saggital midline extends between the patient's nasion and inion.

17. The method of claim 1 wherein the display of the brain scan image includes a scale relating dimensions on the display to dimensions on the patient's head and the computer makes use of the scale to perform the determining step.

18. A method for locating a lesion in the brain of a patient, the method being performed with the aid of lesion location measurement data obtained from a computer system having a display with a representation of a brain scan image illustrating features of the interior of the patient's cranium, the measurement data including a first dimension corresponding to the distance in the representation along a saggital midline on the patients cranium between an anatomic landmark on the patient's head and the lesion, and a second dimension corresponding to the distance, in a coronal view in the representation, along the patient's cranium between the saggital midline and the lesion, the method comprising the steps of:
along a substantially saggital midline on the surface the patient's cranium, laying out the first dimension as a distance from the anatomic landmark, to arrive at a first point on the cranium; and
laying out the second dimension along a line substantially perpendicular to the saggital midline, as a distance from the saggital midline to arrive at a target point corresponding substantially to the location of the lesion.

19. The method of claim 18 wherein the second dimension is measured from the first point.

20. The method of claim 19 wherein the anatomic landmark is the patient's nasion.

21. The method of claim 19 wherein the landmark is the patient's inion.

22. The method of claim 19 wherein the substantially saggital midline extends between the patient's nasion and inion.

23. The method of claim 18 further comprising establishing a substantially coronal midline on the surface of the patient's cranium as a line extending between the patient's ear canals, the and being measure on the coronal midline to produce an intermediate point, the intermediate point being aligned with the first point.

24. The method of claim 19 further comprising establishing a substantially coronal midline on the surface of the patient's cranium as a line extending between the patient's ear canals, the and being measure on the coronal midline to produce an intermediate point, the intermediate point being aligned with the first point.

25. The method of claim 18 wherein the display of the representation of the brain scan image includes a scale relating dimensions on the display to dimensions on the patient's head and the computer makes use of the scale to produce the measurement data.
